(19) United States
(12) Patent Application Publication
(10) Pub. No.: US 2025/0345599 A1
(43) Pub. Date: Nov. 13, 2025

(54) MEDICAL LEAD SYSTEM

(71) Applicant: MEDTRONIC, INC., Minneapolis, MN (US)

(72) Inventors: Kevin R. Seifert, Forest Lake, MN (US); Mark T. Marshall, Cape Coral, FL (US); Claire E. Rydeen, Minneapolis, MN (US); Thomas A. Wong, Minneapolis, MN (US); Pradipta K. Das, Plymouth, MN (US); Andrew J. Ries, Lino Lakes, MN (US); Jonathan A. Hughes, Blaine, MN (US)

(21) Appl. No.: 18/868,819

(22) PCT Filed: May 30, 2023
(86) PCT No.: PCT/US2023/023808

§ 371 Date: Nov. 25, 2024

Related U.S. Application Data
(60) Provisional application No. 63/347,308, filed on May 31, 2022.

(51) Int. Cl. A61N 1/05; A61N 1/375

(52) U.S. Cl. CPC A61N 1/057; A61N 1/37518; A61N 2001/058

(57) ABSTRACT

Various embodiments of a medical lead system are disclosed. The lead system includes a lead body that includes a lumen that extends along the lead body between an inlet adjacent to a proximal end of the lead body and an outlet adjacent to a distal end of the lead body; a fixation member; and a distal balloon connected to an exterior surface of the distal end of the lead body and fluidly connected to the outlet of the lumen. The medical lead system further includes a pressure sensor fluidly connected to the inlet of the lumen of the lead body and configured to sense a pressure within an interior volume of the distal balloon and communicate a signal indicative of the pressure to a clinician. The fixation member is further configured to extend distal to the distal balloon when the distal balloon is in an inflated configuration.

### CROSS-REFERENCE TO RELATED APPLICATIONS

This application claims the benefit of U.S. Provisional Application No. 63/347,308, filed May 31, 2022, the disclosure of which is incorporated by reference herein in its entirety.

### TECHNICAL FIELD

This disclosure generally relates to an implantable lead system, such as a medical lead for implantation in a heart.

### BACKGROUND

Implantable medical leads may be configured to treat a wide variety of cardiac dysfunctions and can include one or more electrodes and other elements for physiological sensing or therapy delivery. Such leads can allow the electrodes to be positioned at one or more target locations for those functions. An implantable medical lead can be navigated through vasculature of a patient to reach these target locations. An electrode supported by the medical lead may establish electrical communication with tissue of the heart to sense cardiac signals generated by the heart and deliver cardiac pacing to the patient. Evaluation of the target location is often conducted to determine a satisfactory location for the electrode within the heart to provide adequate activation of the cardiac system.

### SUMMARY

The techniques of this disclosure generally relate to a medical lead system and an implantable medical device assembly that includes such lead system. The system can include a lead having a distal balloon connected to an exterior surface of a distal end of a lead body of the lead. The distal balloon can be fluidly connected to an outlet of a lumen of the lead. The system can also include a pressure sensor that is fluidly connected to an inlet of the lumen of the lead. The pressure sensor can be configured to sense pressure within an interior volume of the distal balloon and communicate a signal that is indicative of such pressure to a clinician. In one or more embodiments, the system can also include an inflation mechanism configured to be fluidly connected to the inlet of the lumen of the lead body and manipulate the distal balloon between the deflated configuration and the inflated configuration. The pressure sensor can include any suitable sensor. In one or more embodiments, the pressure sensor can include an indicator balloon that defines a deflated configuration and an inflated configuration. The indicator balloon can be in the deflated configuration when the distal balloon is in the deflated configuration. Further, the indicator balloon can be in the inflated configuration when the distal balloon is in the inflated configuration.

This disclosure includes without limitation the following clauses:

Clause 1: A medical lead system that includes a lead. The lead includes a lead body that has a distal end configured to extend through vasculature to a chamber of a heart of a patient, where the lead body further includes a lumen that extends along the lead body between an inlet adjacent to a proximal end of the lead body and an outlet adjacent to the distal end of the lead body; a fixation member configured to extend distal to the distal end of the lead body, where the fixation member is configured to be secured to tissue of the heart; and a distal balloon connected to an exterior surface of the distal end of the lead body and fluidly connected to the outlet of the lumen, where the distal balloon defines a deflated configuration and an inflated configuration, and further where the distal balloon is configured to expand radially outward from the exterior surface of the lead body and extend distally beyond the distal end of the lead body when the distal balloon inflates from the deflated configuration to the inflated configuration. The medical lead system further includes a pressure sensor fluidly connected to the inlet of the lumen of the lead body and configured to sense a pressure within an interior volume of the distal balloon and communicate a signal indicative of the pressure to a clinician. The fixation member is further configured to extend distal to the distal balloon when the distal balloon is in the inflated configuration.

Clause 2: The system of Clause 1, further including an inflation mechanism configured to be fluidly connected to the inlet of the lumen of the lead body and manipulate the distal balloon between the deflated configuration and the inflated configuration.

Clause 3: The system of Clause 2, where the inflation mechanism includes a syringe or a pump.

Clause 4: The system of any one of Clauses 2-3, where the pressure sensor is connected to the inflation mechanism.

Clause 5: The system of any one of Clauses 1-4, where the pressure sensor includes an indicator balloon that defines a deflated configuration and an inflated configuration, where the indicator balloon is in the deflated configuration when the distal balloon is in the deflated configuration, and further where the indicator balloon is in the inflated configuration when the distal balloon is in the inflated configuration.

Clause 6: The system of any one of Clauses 1-4, where the pressure sensor includes a disk and a base configured to receive the disk, where a major surface of the disk faces a major surface of the base, where the signal includes a first configuration and a second configuration of the disk, where a first distance between a center portion of the major surface of the disk and the major surface of the base when the disk is in the first configuration as measured along a normal to the major surface of the disk and major surface of the base is less than a second distance between the center portion of the major surface of the disk and the major surface of the base when the disk is in the second configuration. The disk is in the first configuration when the distal balloon is in the deflated configuration and in the second configuration when the distal balloon is in the inflated configuration.

Clause 7: The system of any one of Clauses 1-4, where the pressure sensor includes a pressure transducer.

Clause 8: The system of any of Clauses 1-4, where the pressure sensor includes an audible alarm.

Clause 9: The system of any one of Clauses 1-4, where the pressure sensor includes an indicator light, where the indicator light is configured to emit light including a first wavelength when the distal balloon is in the deflated configuration and emit light including a second wavelength when the distal balloon is in the inflated configuration.

Clause 10: The system of any one of Clauses 1-4, where the pressure sensor includes a colored diaphragm and a frosted lens, where the colored diaphragm is configured to move in a direction normal to the diaphragm and frosted lens. A contact area between the colored diaphragm and the frosted lens corresponds to a pressure within the interior volume of the distal balloon.

Clause 11: The system of any one of Clauses 1-4, where the pressure sensor includes a mechanical switch, where the signal includes a change of shape of the mechanical switch.

Clause 12: An implantable medical device assembly that includes an implantable medical device having a housing and an electronics module disposed within the housing and including a power source and circuitry electrically connected to the power source. The assembly further includes a lead system. The lead system includes a lead that includes a lead body having a distal end configured to extend through vasculature to a chamber of a heart of a patient, where the lead body includes a lumen that extends along the lead body between an inlet adjacent to a proximal end of the lead body and an outlet adjacent to the distal end of the lead body, where at least a proximal portion of the lead body is configured to be inserted into a lead bore of the housing; a fixation member configured to extend distal to the distal end of the lead body, where the fixation member is configured to secure to tissue of the heart; and a distal balloon connected to an exterior surface of the distal end of the lead body and fluidly connected to the outlet of the lumen, where the distal balloon defines a deflated configuration and an inflated configuration, and further where the distal balloon is configured to expand radially outward from the exterior surface of the lead body and extend distally beyond the distal end of the lead body when the distal balloon inflates from the deflated configuration to the inflated configuration. The lead system further includes a pressure sensor fluidly connected to the inlet of the lumen of the lead body and configured to sense a pressure within an interior volume of the distal balloon and communicate a signal indicative of the pressure to a clinician. The fixation member is configured to extend distal to the distal balloon when the distal balloon is in the inflated configuration.

Clause 13: The assembly of Clause 12, wherein the lead bore of the implantable medical device comprises a contact that is electrically connected to the circuitry of the electronics module by a conductor, wherein a lead contact of the lead is configured to be electrically connected to the contact of the lead bore when the at least a portion of the lead is disposed within the lead bore.

Clause 14: The assembly of any one of Clauses 12-13, where the implantable medical device is an implantable defibrillator.

Clause 15: The assembly of any one of Clauses 12-13, where the implantable medical device is a neuromodulation device.

Clause 16: The assembly of any one of Clauses 12-15, further including a desiccant disposed within the housing.

Clause 17: The assembly of any one of Clauses 12-16, further including an inflation mechanism configured to be fluidly connected to the inlet of the lumen and manipulate the distal balloon between the deflated configuration and the inflated configuration.

Clause 18: The assembly of Clause 17, where the inflation mechanism includes a syringe or a pump.

Clause 19: The assembly of any one of Clauses 17-18, where the pressure sensor is connected to the inflation mechanism.

Clause 20: The assembly of any one of Clauses 12-19, where the pressure sensor includes an indicator balloon that defines a deflated configuration and an inflated configuration, where the indicator balloon is in the deflated configuration when the distal balloon is in the deflated configuration. The indicator balloon is in the inflated configuration when the distal balloon is in the inflated configuration.

Clause 21: The assembly of any one of Clauses 12-19, where the pressure sensor includes a disk and a base configured to receive the disk. A major surface of the disk faces a major surface of the base. The disk includes a first configuration and a second configuration. A first distance between a center portion of the major surface of the disk and the major surface of the base when the disk is in the first configuration as measured along a normal to the major surface of the disk and major surface of the base is less than a second distance between the center portion of the major surface of the disk and the major surface of the base when the disk is in the second configuration. The disk is in the first configuration when the distal balloon is in the deflated configuration and in the second configuration when the distal balloon is in the inflated configuration.

Clause 22: The assembly of any one of Clauses 12-19, where the pressure sensor includes a pressure transducer.

Clause 23: The assembly of any one of Clauses 12-19, where the pressure sensor includes an audible alarm.

Clause 24: The assembly of any one of Clauses 12-19, where the pressure sensor includes an indicator light. The indicator light is configured to emit light including a first wavelength when the distal balloon is in the deflated configuration and emit light including a second wavelength when the distal balloon is in the inflated configuration.

Clause 25: The assembly of any one of Clauses 12-19, where the pressure sensor includes a colored diaphragm and a frost lens. The colored diaphragm is configured to move in a direction normal to the diaphragm and frosted lens, wherein an area of contact between the colored diaphragm and the frosted lens corresponds to the pressure of the distal balloon.

Clause 26: The assembly of any one of Clauses 12-19, where the pressure sensor includes a mechanical switch. The signal includes a change of shape of the mechanical switch.

Clause 27: A method that includes inflating a distal balloon from a deflated configuration to an inflated configuration by expanding the distal balloon radially outward from an exterior surface of a lead body configured to extend through vasculature to a chamber of a heart of a patient; extending the distal balloon distally beyond a distal end of the lead body and proximal to a fixation member when the distal balloon inflates from the deflated configuration to the inflated configuration; and sensing a pressure within an interior volume of the distal balloon. The method further includes communicating a signal to a clinician that is indicative of the pressure within the interior volume of the distal balloon.

Clause 28: The method of Clause 27, further including substantially surrounding a portion of the fixation member with the distal balloon when the distal balloon inflates to the inflated configuration.

Clause 29: The method of any one of Clauses 27-28, where the lead body defines a lumen fluidly connected to the interior volume of the distal balloon at the distal end of the lead body. The lumen extends along the lead body between an inlet adjacent to a proximal end of the lead body and an outlet adjacent to the distal end of the lead body.

Clause 30: The method of Clause 29, where inflating the distal balloon includes connecting an inflation mechanism to the inlet of the lead body.

Clause 31: The method of any one of Clauses 29-30, where sensing the pressure includes fluidly connecting a pressure sensor to the lumen at the proximal end of the lead body, where communicating the signal indicative of the pressure includes communicating the signal indicative of the pressure within the interior volume of the distal balloon to the clinician utilizing the pressure sensor.

Clause 32: The method of Clause 31, where the signal includes a visual signal.

Clause 33: The method of Clause 32, where the pressure sensor includes an indicator balloon, where the visual signal includes an inflation volume of the indicator balloon.

Clause 34: The method of Clause 32, where the pressure sensor includes a pressure transducer.

Clause 35: The method of Clause 32, where the pressure sensor includes an indicator light.

Clause 36: The method of Clause 32, where the pressure sensor includes a disk and a base configured to receive the disk. A major surface of the disk faces a major surface of the base, where the signal includes a first configuration and a second configuration of the disk. A first distance between a center portion of the major surface of the disk and the major surface of the base when the disk is in the first configuration as measured along a normal to the major surface of the disk and major surface of the base is less than a second distance between the center portion of the major surface of the disk and the major surface of the base when the disk is in the second configuration. The disk is in the first configuration when the distal balloon is in the deflated configuration and in the second configuration when the distal balloon is in the inflated configuration.

Clause 37: The method of Clause 31, where the signal includes an audible signal.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram of one embodiment of an implantable medical device assembly accessing a target site within a patient.

FIG. 2 is a schematic cross-section view of one embodiment of a medical lead system and implantable medical device of the implantable medical device assembly of FIG. 1.

FIG. 3 is a schematic perspective view of the implantable medical device of FIG. 2

FIG. 4 is a schematic perspective view of the implantable medical device of FIG. 2 with a cover removed for clarity.

FIG. 5 is a schematic cross-section view of a portion of the medical lead system of FIG. 1 disposed within a sheath.

FIG. 6 is a schematic cross-section view of a portion of the medical lead system of FIG. 1 positioned distal to the sheath.

FIG. 7 is a schematic cross-section view of a portion of the medical lead system of FIG. 1 with a distal balloon of the system in an inflated configuration.

FIG. 8 is a schematic cross-section view of a portion of the medical lead system of FIG. 1 in the vicinity of a tissue wall.

FIG. 9 is a schematic cross-section view of a portion of the medical lead system of FIG. 1 in the vicinity of a target site on the tissue wall.

FIG. 10 is a schematic cross-section view of a portion of the medical lead system of FIG. 1 with the distal balloon in a deflated configuration.

FIG. 11 is a schematic cross-section view of a portion of the medical lead system of FIG. 1 with the distal balloon in the inflated configuration.

FIG. 12 is flowchart of one embodiment of a method of using the medical lead system of FIG. 1.

FIG. 13 is a schematic cross-section view of another embodiment of a pressure sensor in a deflated configuration.

FIG. 14 is a schematic cross-section view of the pressure sensor of FIG. 13 in an inflated configuration.

FIG. 15 is a schematic cross-section view of another embodiment of a pressure sensor in a first configuration.

FIG. 16 is a schematic cross-section view of the pressure sensor of FIG. 15 in a second configuration.

FIG. 17 is a schematic cross-section view of another embodiment of a pressure sensor in a first configuration.

FIG. 18 is a schematic plan view of the pressure sensor of FIG. 17 in the first configuration.

FIG. 19 is a schematic cross-section view of the pressure sensor of FIG. 17 in a second configuration.

FIG. 20 is a schematic cross-section view of another embodiment of a pressure sensor.

FIG. 21 is a schematic cross-section view of another embodiment of a pressure sensor.

FIG. 22 is a schematic perspective view of another embodiment of a pressure sensor.

FIG. 23 is a schematic cross-section view of another embodiment of a pressure sensor.

FIG. 24 is a flowchart of one embodiment of a method of sensing a pressure of the distal balloon of the medical lead system of FIG. 1.

FIG. 25 is a graph of pressure within an interior volume of an exemplary distal balloon versus time.

### DETAILED DESCRIPTION

The techniques of this disclosure generally relate to a medical lead system and an implantable medical device assembly that includes such lead system. The system can include a lead having a distal balloon connected to an exterior surface of a distal end of a lead body of the lead. The distal balloon can be fluidly connected to an outlet of a lumen of the lead. The system can also include a pressure sensor that is fluidly connected to an inlet of the lumen of the lead. The pressure sensor can be configured to sense pressure within an interior volume of the distal balloon and communicate a signal that is indicative of such pressure to a clinician. In one or more embodiments, the system can also include an inflation mechanism configured to be fluidly connected to the inlet of the lumen of the lead body and manipulate the distal balloon between the deflated configuration and the inflated configuration. The pressure sensor can include any suitable sensor. In one or more embodiments, the pressure sensor can include an indicator balloon that defines a deflated configuration and an inflated configuration. The indicator balloon can be in the deflated configuration when the distal balloon is in the deflated configuration. Further, the indicator balloon can be in the inflated configuration when the distal balloon is in the inflated configuration.

The medical lead system can be configured to transit through vasculature of the patient to position a distal portion of the lead body of the lead in the vicinity of a target area, such as an area within a chamber of the heart. For example, the medical lead system can be configured to allow a clinician to navigate the lead body through a vein of the heart (e.g., at least one of an innominate vein, an interior vena cava (IVC), or a superior vena cava (SVC)) to a target location within a right ventricle (RV), right atrium (RA), or another area of the heart. In one or more embodiments, the medical lead system is configured such that the distal balloon can be inflated within a vein of the heart enroute to the target area to, for example, minimize or eliminate physical interference between the fixation member and other anatomical structures as the medical lead system is transited within the patient.

The clinician may find it difficult to observe whether the distal balloon is the deflated configuration or the inflated configuration as it is disposed on the distal end of the lead when the lead is disposed within the patient's body, especially without the use of fluoroscopy or other imaging techniques. As a result, the clinician may not receive any feedback from the distal balloon as to whether it is in the inflated or deflated configuration. Further, the distal balloon may develop a leak while the lead is being advanced within the patient. Such leak would potentially go undetected by the clinician. If the distal balloon is not fully inflated as the lead is advanced within the patient, physical interference between the fixation member of the lead and other anatomical structures can occur.

One or more embodiments of the present disclosure can provide various advantages over currently available lead systems. For example, the pressure sensor of the medical lead system can be configured to sense the pressure within the interior volume of the distal balloon and communicate the signal indicative of such pressure to the clinician. Such signal can, for example, indicate to the clinician whether the distal balloon is the inflated or deflated configuration, or whether the balloon is losing pressure due to a leak. The pressure sensor can include any suitable type of pressure sensor, e.g., at least one of a mechanical, electrical, auditory, or visual sensor. Further, one or more embodiments of a medical lead system of the present disclosure can include an inflation mechanism that is configured to be fluidly connected to the inlet of the lumen of the lead body and manipulate the distal balloon between the deflated configuration and the inflated configuration.

In general, the distal balloon can be configured to expand radially outward from the lead body when the balloon is inflated from the deflated configuration to the inflated configuration. Further, in one or more embodiments, the distal balloon can be configured to define a substantially toroidal shape around the lead body in the inflated condition such that, for example, the lead body extends through the toroid hole. In one or more embodiments, the fixation member is configured to extend through the toroid hole. In one or more embodiments, the distal balloon is configured to expand around the distal portion of the lead body such that a blood flow imparts a force on the balloon in the direction of the blood flow (e.g., such that the balloon acts as a “sail”). The distal balloon can be configured to transmit the force to the lead body to assist in transiting the lead body through the vasculature and/or a heart chamber of the patient while substantially shrouding the fixation member. In one or more embodiments, the distal balloon is configured to inflate to an expanded dimension (e.g., a diameter) of at least twenty times a diameter of the lead body. In one or more embodiments, the lead body defines an inner lumen configured to deliver an inflating medium to cause inflation of the distal balloon.

The medical lead system can be configured to electrically communicate with tissue within a patient when the distal balloon is in the inflated configuration. In one or more embodiments, the fixation member mechanically supports an electrode configured to electrically communicate with the tissue. The fixation member can be configured to position the electrode to communicate with the tissue when the fixation member extends distal to the inflated balloon. In one or more embodiments, the fixation member is configured to allow the electrode to contact the tissue when the fixation member extends distal to the inflated balloon. The medical lead system can include a conductor in electrical communication with the electrode. In one or more embodiments, the medical lead system includes processing circuitry configured to deliver therapy to the tissue using the conductor and the electrode. The medical lead system may be configured such that a clinician can perform cardiac mapping using the electrode while the balloon is inflated, such that the cardiac mapping may occur as the balloon substantially shrouds the fixation member to reduce and/or eliminate physical interferences between the fixation member and surrounding anatomical structures.

The distal balloon is configured to deflate from the inflated configuration to substantially re-establish the deflated configuration. For example, a clinician may cause the balloon to substantially re-establish the deflated configuration (e.g., by venting the balloon through the lumen) to secure the medical lead system to the tissue using the fixation member. In one or more embodiments, the fixation member defines a helix extending distal to the lead body distal end and at least partially shrouded by the distal balloon. The clinician may deflate the balloon via the lumen to cause the balloon to deflate and withdraw proximal to the lead body distal end, such that the fixation member is substantially unshrouded. The clinician may then cause the helix to substantially engage the tissue by, for example, torquing the lead body around a longitudinal axis to drive the helix into a surface of the tissue. The distal balloon may be configured to remain affixed to the lead body and proximal to the fixation member and/or electrode in the deflated configuration, such that processing circuitry may deliver therapy to the patient via the electrode with the balloon in the deflated configuration.

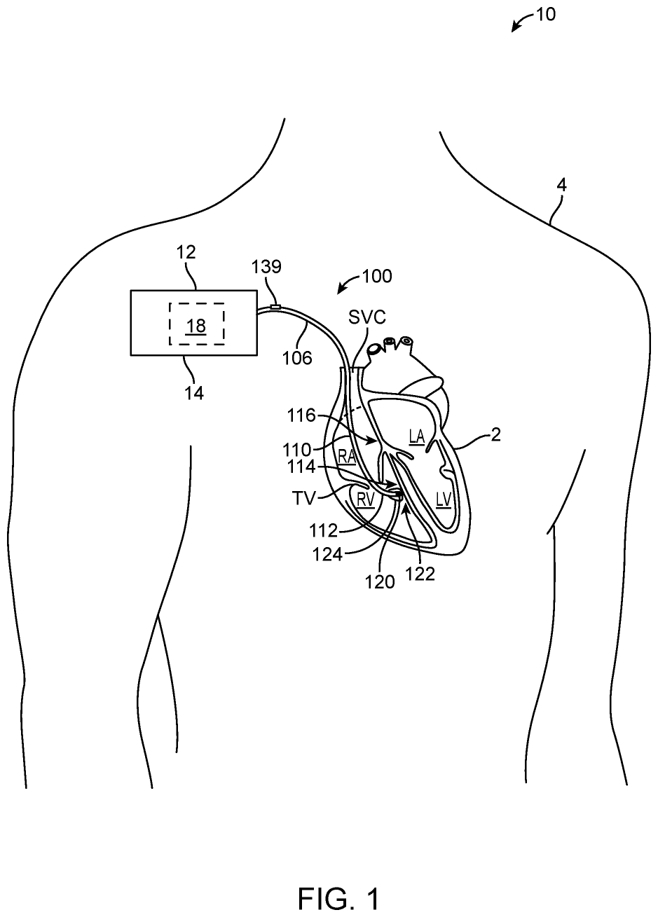

FIGS. 1-11 are various views of one embodiment of an implantable medical device assembly 10 that includes an implantable medical device 12 and a medical lead system 100. The assembly 10 can be configured to deliver therapy (e.g., pacing) to a heart 2 of a patient 4.

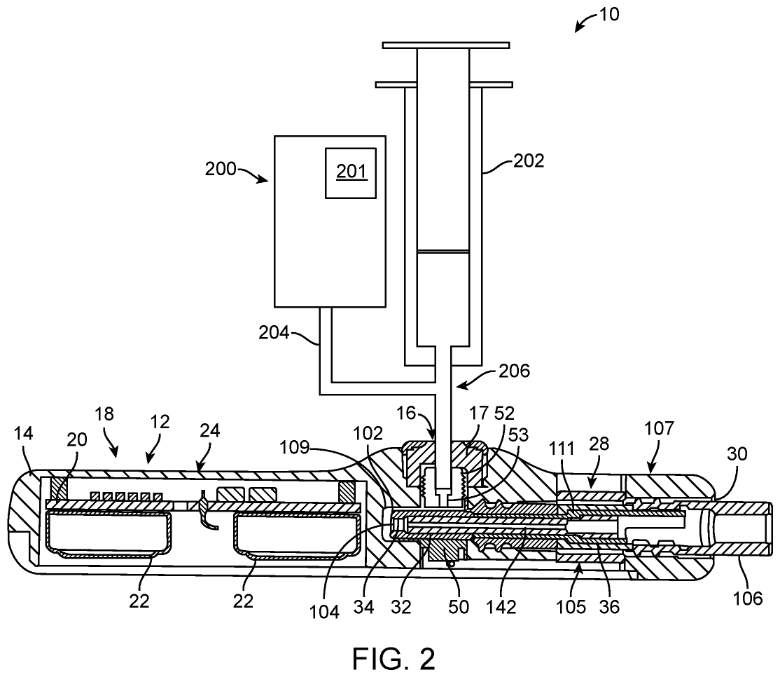

The implantable medical device 12 includes a housing 14 and an electronics module 18 disposed within the housing. The electronics module 18 can include a power source 22 and circuitry 24 electrically connected to the power source (FIG. 2).

The medical lead system 100 can include any suitable lead system, e.g., one or more embodiments of medical lead systems described in co-owned U.S. Patent Application Ser. No. 63/213,674, filed Jun. 22, 2021, and entitled MEDICAL LEAD SYSTEM. The medical lead system 100 of the assembly 10 includes a lead 106 having a lead body 110 that includes a distal portion 112 that is generally positioned at a target site 114 within the patient 4. As illustrated in FIG. 1, the target site 114 is a region in the ventricular septal wall of heart 2. In one or more embodiments, the medical lead 106 can be oriented such that the distal portion 112 is disposed at another portion of the heart 2. For example, the lead 106 may be oriented such that the distal portion 112 is generally disposed at a target site 116 in the atrioventricular septal wall. Medical lead system 100 can include additional leads coupled to the medical device 12 and extending into heart 2.

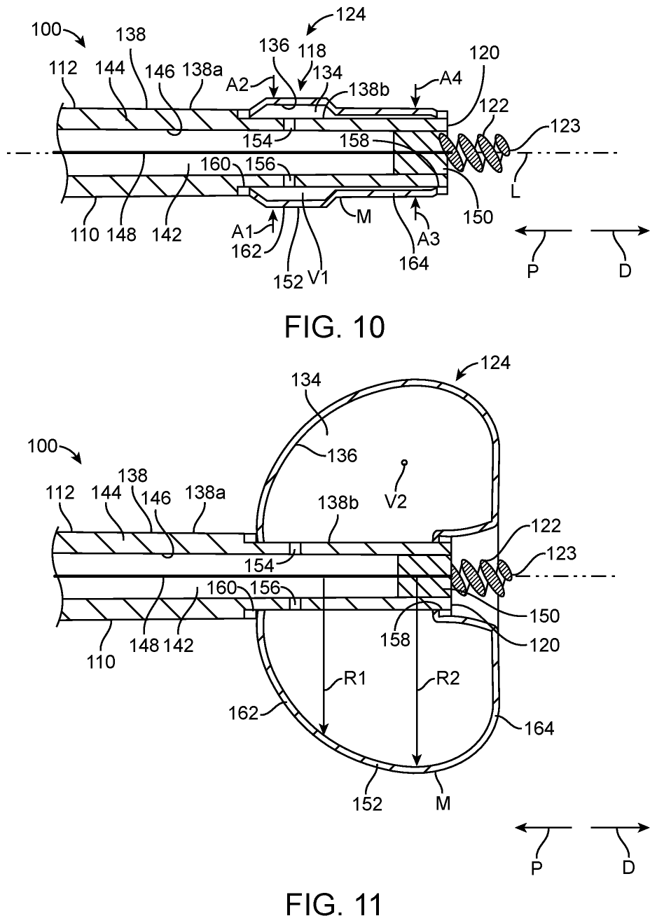

The distal portion 112 of the lead 106 includes a distal end 120 that is configured to extend through vasculature to a chamber of the heart 2. The lead body 110 further includes a lumen 142 (FIG. 2) that extends along the lead body between an inlet 102 adjacent to a proximal end 104 of the lead body, and an outlet 118 (FIG. 10) adjacent to a distal end 120 of the leady body. As used herein, the phrase “adjacent to the proximal end” means that an element or component is disposed closer to the proximal end 104 of the lead body 110 than to the distal end 120. As further used herein, the phrase “adjacent to the distal end” means that an element or component is disposed closer to the distal end 120 of the lead body 110 than to the proximal end 104. At least a proximal portion 105 of the lead body 110 is configured to be inserted into a lead bore 30 of the housing 14 of the medical device 12.

The medical lead system 100 also includes a fixation member 122 configured to extend distal to the distal end 120 of the lead body 110. The fixation member 122 is configured to secure to tissue of the heart 2.

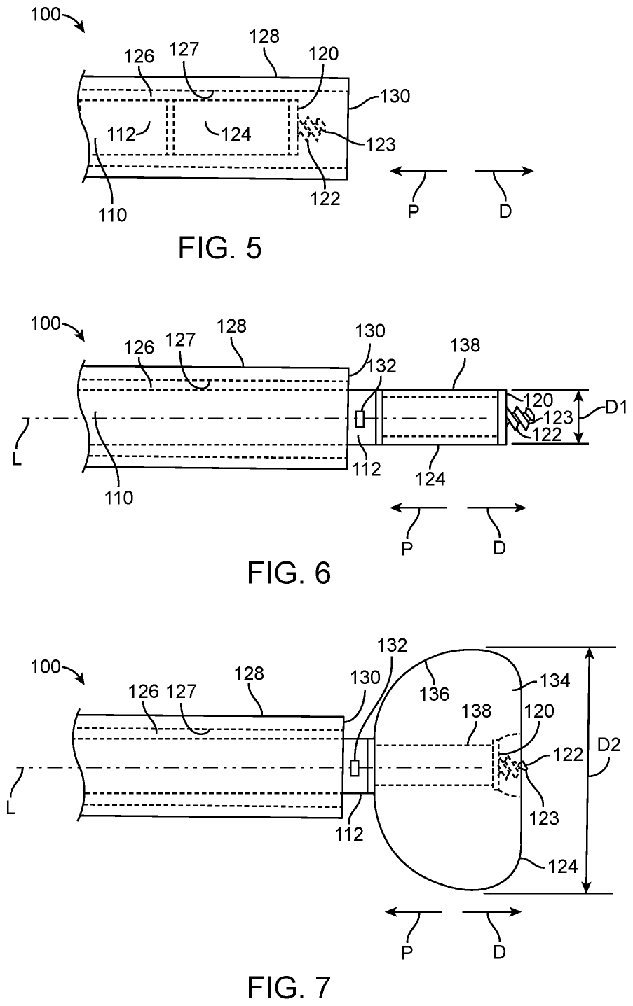

In addition, the lead system 10 includes a distal balloon 124 (FIG. 7) connected to an exterior surface 138 of the distal end 120 of the lead body 110 and that is fluidly connected to the outlet 118 of the lumen 142. As used herein, the phrase “fluidly connected” means that two or more elements or components are connected using any suitable technique such that a fluid or fluids can be directed from one element or component to another element or component via channels or paths that extend between the elements or components. The distal balloon 124 defines a deflated configuration (FIGS. 5-6) and an inflated configuration (FIG. 7). The distal balloon 124 is configured to expand radially outward from the exterior surface 138 of the lead body 110 and extend distally beyond the distal end 120 of the lead body when the distal balloon inflates from the deflated configuration to the inflated configuration. The fixation member 122 is configured to extend distal to the distal balloon 124 when the distal balloon is in the inflated configuration.

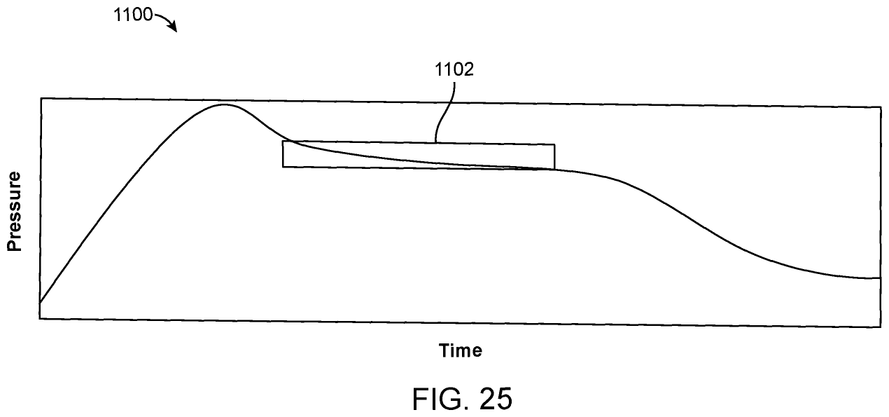

Also included in the medical lead system 100 is a pressure sensor 200 (FIG. 2) fluidly connected to the inlet 102 of the lumen 142. The pressure sensor 200 is configured to sense a pressure within an interior volume 134 of the distal balloon 124 and communicate a signal indicative of the pressure to a clinician. In one or more embodiments, the pressure sensor 200 can include a processor 201 that can be configured to monitor the pressure within the interior volume 134 of the distal balloon 124 over time. For example, FIG. 25 is a graph 1100 of pressure within the interior volume 134 of the distal balloon 124 versus time. The pressure sensor 200 can be configured to monitor this pressure over time via the processor 201 and determine a rate of change of such pressure, and determine whether the pressure stays within a desired range 1102 as shown in FIG. 25. In one or more embodiments, the processor 201 can be configured to manipulate an inflation mechanism 202 to control the pressure within the interior volume 134 of the distal balloon 124. Any suitable technique can be utilized by the processor 201 to control the inflation mechanism 202.

The pressure sensor 200 can be fluidly connected to the inlet 102 of the lumen 142 using any suitable technique. In one or more embodiments, the pressure sensor 200 can be directly connected to the inlet 102 by one or more conduits 204. Such conduit 204 can be configured to direct inflating medium from at least one of the lumen 142 and the inflation mechanism 202 to the sensor 200. A fluid path 206 can be formed between the sensor 200, the lumen 142, and the distal balloon 124 through the outlet 118 that is closed and exhibits a pressure that can be measured by the pressure sensor 200.

In one or more embodiments, the medical lead system 100 further includes the inflation mechanism 202 configured to be fluidly connected to the inlet 102 of the lumen 142 and manipulate the distal balloon 124 between the deflated configuration and the inflated configuration. The fluid path 206 can include the inflation mechanism 202 when the mechanism is fluidly connected to the inlet 102 of the lumen 142. The inflation mechanism 202 can include any suitable device such as a syringe or a pump that can provide an inflating medium to the distal balloon 124 via the lumen 142. The inflation mechanism 202 can be connected to the device 12 using any suitable technique. As shown in FIG. 2, the mechanism 202 is connected to a port 16 of the device 12 such that the mechanism is fluidly connected to the inlet 102 of the lumen 142. The inflating medium that is provided to the distal balloon 124 can include any suitable gas or liquid, e.g., air.

The implantable medical device 12 of the system 10 can include any suitable medical device that is configured to be implanted within a body of a patient. In one or more embodiments, the device 12 can be a pacemaker. Further, in one or more embodiments, the device 12 can be a leadless cardiac monitor. The device 12 can include any other suitable medical devices such as at least one of a defibrillator, LVAD, neuromodulation device, or drug pump, or other implantable medical device.

The housing 14 of the device 12 can take any suitable shape or shapes and have any suitable dimensions. Further, the housing 14 can include any suitable material or materials, e.g., at least one of a metallic, polymeric, or inorganic material. Suitable materials for the housing 14 can include at least one of titanium (e.g., any suitable grade such as grade 5 titanium), stainless steel, polymer, ceramic, glass, or combinations thereof such as laminates, composites, or miscible blends or mixtures. In one or more embodiments, the housing 14 can include any suitable polymeric material or materials, e.g., at least one of epoxy, polyurethane, silicone, polyolefin, acrylic polymer, polyester, polyether ether ketone (PEEK), polysulfone, polymethylene oxide, or polyvinyl, or combinations thereof.

The housing 14 can be a unitary housing. In one or more embodiments, the housing 14 can include two or more portions that are connected using any suitable technique or techniques, e.g., welding, mechanically fastening, adhering, thermal bonding, diffusion bonding, laser-assisted diffusion bonding, solvent bonding, over-molding, etc. Further, the housing 14 can be formed using any suitable technique or techniques, e.g., molding, thermoforming, laminating, over-molding, casting, insert molding, etc.

The housing 14 can include any suitable ports or receptacles that can connect the device to external components or systems. For example, the housing 14 can include the lead bore 30 disposed in any suitable portion or portions of the housing. The lead bore 30 can take any suitable shape or shapes and have any suitable dimensions. The housing 14 can also include a setscrew block 50 disposed in any suitable portion or portions of the housing that is configured to receive a setscrew 52 that includes a bore or lumen 53 for directing inflating fluid from the inflation mechanism 202 to the lumen 142 of the lead 14. The setscrew block 50 can take any suitable shape or shapes and have any suitable dimensions. The setscrew block 50 can receive the setscrew 52 that is configured to retain the lead 14 in the lead bore 30. The port 16 can be disposed through a silicone grommet 17 through which the inflating medium can be provided to the distal balloon 124 using the inflation mechanism 202. The port 16 can be disposed in any suitable portion of the housing 14. In one or more embodiments, the inflating medium can be delivered by inserting the inflation mechanism 202 into the sealing grommet 17 and the bore 53 such that the inflating medium can pass through the bore and into the lumen 142 of the lead 14. In one or more embodiments, a gap can be formed between the bore 53 of the setscrew 52 and the lead 14 such that the inflating medium can exit the bore and be directed into the lead bore 30 where it can be further directed into the inlet 102 of the lumen 142.

The implantable medical device 12 can further include any suitable materials disposed within the housing 14. For example, a desiccant can be disposed in any suitable location within the housing 14. The desiccant can include any suitable material that can absorb moisture present within the housing 14, e.g., at least one of a molecular sieve, silica gel, or a combination of silicone elastomer mixed with one of the desiccant materials.

Further, one or more polymeric filler materials can be disposed in any suitable location within the housing 14. The polymeric filler material can include any suitable material or materials, e.g., at least one of a medical adhesive, a medical grade silicone, an epoxy, or a polyurethane.

Implantable medical device 12 can further include electronic circuitry contained within the housing 14, where the circuitry may be configured to deliver cardiac pacing. As shown in FIG. 2, the electronics module 18 can include a substrate 20, the power source 22 disposed on the substrate, and the circuitry 24 also disposed on the substrate and electrically connected to the power source. The power source 22 can include any suitable power source or sources, e.g., one or more batteries. Although depicted as including two power sources, the power source 22 can include any suitable number of power sources, e.g., one, two, three, four, five, or more power sources. The power source 22 can be disposed on any suitable portion or portions of the substrate 20. In one or more embodiments, the power source 22 can be disposed in the housing 14 in any suitable location without being disposed on a substrate.

Also disposed on the substrate 20 is the circuitry 24. Such circuitry 24 can include any suitable device or component, e.g., at least one of a capacitor, transistor, integrated circuit including a controller or multiplexer, sensor, accelerometer, inductive charging coil, antenna, optical components such as emitters and detectors, etc. Such components can be electrically connected to the power source 22 or one or more components using any suitable technique or techniques. Further, the circuitry 24 can be disposed on any suitable portion of the substrate 20. In one or more embodiments, one or more components of the circuitry 24 can be disposed apart from one or more additional components of the circuitry within or on the housing 14 and electrically connected to the power source 22 and one or more additional components using any suitable technique or techniques. In one or more embodiments, the circuitry 24 and the power source 22 can be coated with an intermediary barrier to form a conformal coating. Any suitable technique can be utilized. For example, chemical vapor deposition can be utilized to provide a coating of parylene or any other material that provides a protective barrier to the circuitry 24 and the power source 22.

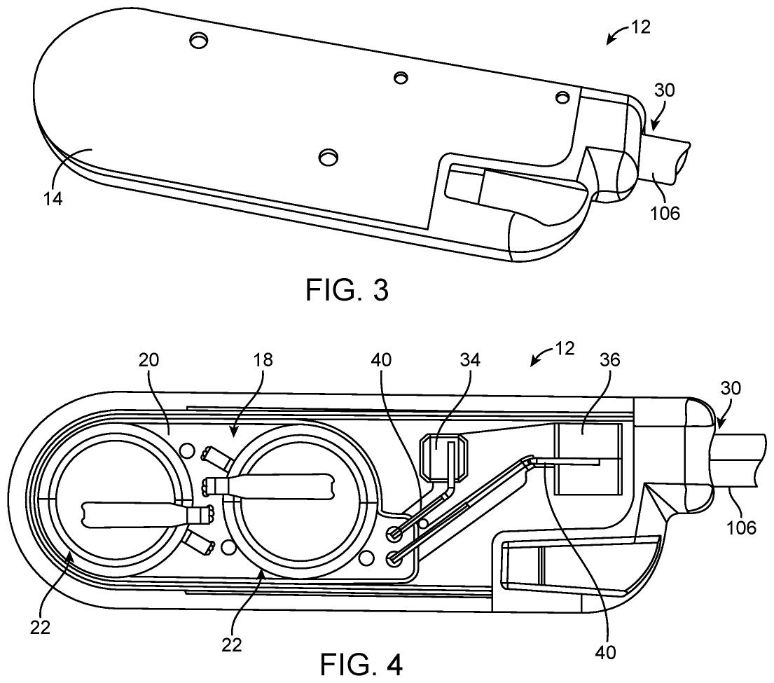

The assembly 10 also includes the medical lead system 100 that has the lead 106 that is configured to be electrically connected to the electronics module 18 disposed in the housing 14 of the device 12. The lead 106 can include any suitable lead, e.g., pacing, defibrillation, or nerve stimulation lead in an industry standard or custom format, etc. Although depicted as including one lead 106, the system 100 can include any suitable number of leads that are electrically connected to the electronics module 18. The lead 106 can be electrically connected to the electronics module 18 using any suitable technique or techniques. In one or more embodiments, at least a proximal portion 107 of the lead 106 can be disposed within the lead bore 30 of the housing 14 such that a lead contact 109 is electrically connected to contact 34 disposed within the lead bore 30 when the proximal portion of the lead is disposed within the lead bore. In one or more embodiments, the lead 106 can include a second lead contact 111 that is configured to electrically connect the lead to a second contact 36 disposed within the lead bore 30 when the proximal portion 107 of the lead is disposed within the lead bore. The contact 34 and the second contact 36 can be electrically connected to the electronics module 18 by conductors 40 as is shown in FIG. 3.

As mentioned herein, the lead 106 includes the fixation member 122, which is mechanically supported by the distal portion 112 of the lead. Fixation member 122 is configured to penetrate tissue of the patient 4 at or near target sites, such as target sites 114, 116. For example, fixation member 122 may be configured to penetrate cardiac tissue of a septal wall in a right ventricle (RV), right atrium (RA), left ventricle (LV), and/or left atrium (LA) of heart 2, or penetrate cardiac tissue in another area of heart 2. In one or more embodiments, fixation member 122 is configured to remain substantially stationary with respect to the distal end 120 of the lead 106 such that the fixation member is substantially fixed in place on the distal portion 112 of the lead body. In one or more embodiments, the fixation member 122 may be configured to translate relative to the distal end 120 of the lead 106. For example, the fixation member 122 may be configured to translate distally or proximally within a lumen defined by the distal portion 112. Fixation member 122 may have various shapes such as helices, tines, screws, rings, and so on.

In one or more embodiments, the fixation member 122 mechanically supports an electrode 123 configured to electrically communicate with tissue when the fixation member positions the electrode in proximity to target sites 114, 116. In one or more embodiments, the electrode is configured to provide pacing to heart 2. The electrode may be electrically connected to one or more conductors 148 extending through the lead body 110. In one or more embodiments, the conductors are electrically connected to the circuitry 24 of medical device 12, with the circuitry configured to deliver therapy signals to and/or sense cardiac signals from the electrode using the conductor. The fixation member 122 can be configured to position the electrode such that the electrode conducts the electrical signals to the target tissue of heart 2, causing the cardiac muscle, e.g., of the ventricles, to depolarize and, in turn, contract at a regular interval.

Medical lead system 100 also includes the distal balloon 124 attached to the exterior surface 138 of the distal end 120 of the lead body 110. The distal balloon 124 defines an interior volume 134 configured to receive an inflating medium (e.g., air, saline, or another medium) and cause inflation of the balloon 124. In one or more embodiments, the lead body 110 defines the lumen 142 that is fluidly coupled to the interior volume and configured such that the clinician can deliver the inflating medium to the interior volume 134 defined by the distal balloon 124. The distal balloon 124 is configured to inflate from the deflated configuration defining an initial dimension (e.g., an initial diameter) and expand radially outward from distal end 120 of the lead body 110 to the inflated configuration defining an expanded dimension (e.g., an expanded diameter). The distal balloon 124 is depicted in the inflated configuration in FIG. 7. The distal balloon 124 is configured to extend distally beyond the distal end 120 of the lead body 110 when the balloon is in the inflated configuration. In one or more embodiments, the distal balloon 124 defines a substantially toroidal shape surrounding the distal portion 112 of the lead body 110 and the distal end 120 when the balloon is in the inflated configuration. Further, the fixation member 122 (e.g., a distal end of fixation member 122) is configured to extend distal to the distal balloon 124 when the balloon is in the inflated configuration. The distal balloon 124 can be configured to substantially surround a portion of the fixation member 122 in the inflated configuration to, for example, minimize and/or eliminate physical interference between the fixation member and other anatomical structures within the patient.

A clinician may maneuver the lead body 110 through the vasculature of the patient 4 to position the distal portion 112 at or near a target site, such as target site 114, 116. The medical lead system 100 can be configured to allow a clinician to maneuver the lead body 110 through the vasculature when the distal balloon 124 is in the inflated configuration. For example, with the distal balloon 124 in the inflated configuration, the clinician can guide the distal portion 112 of the lead body 110 through the superior vena cava (SVC), into the RA, and past tricuspid valve (TV) into the RV in order to access target site 114 on the atrioventricular septal wall. The distal balloon 124 may substantially surround a portion of the fixation member 122 to maintain at least a radial displacement between the fixation member and anatomical structures within patient 4 (e.g., the TV, or other structures within heart 2) during the transit of the distal portion. The medical lead system 100 can be configured to accommodate other pathways or techniques to reach target sites within patient 4 with the distal balloon 124 in the inflated configuration. For example, the medical lead system 100 can be configured such that the inflated configuration accommodates transit through an innominate vein, an interior vena cava (IVC), and/or another veinous pathway enroute to a chamber of heart 2.

In one or more embodiments, the radial expansion of the distal balloon 124 around the distal portion 112 of the lead body 110 causes a blood flow to impart a force on balloon in the direction of the blood flow. The distal balloon 124 may thus substantially act as a sail, assisting the clinician during a transit to target site 114, 116. Further, the distal balloon 124 can transmit the force imparted to the distal portion 112 of the lead body 110 to assist in transiting distal portion through vasculature of the patient 4 (e.g., through the heart 2) while substantially shrouding the fixation member 122. For example, in the inflated configuration, blood flow from the RA to the RV of heart 2 may impart a force on the distal balloon 124 that assists the transit of lead body distal portion 112 through the TV of heart 2. The distal balloon 124 can transmit the force to distal portion 112 to assist transition of the distal portion through the TV while substantially shrouding the fixation member 122 to minimize and/or eliminate physical interference between fixation member 122 and the TV.

The medical lead system 100 is configured to conduct mapping (e.g., electrophysiological mapping) of regions of the heart 2 to locate an initial target site (e.g., target site 114, 116). In one or more embodiments, the medical lead system 100 is configured to conduct the mapping when the distal balloon 124 is in the inflated configuration. The fixation member 122 can be configured to position the mechanically supported electrode in sufficient proximity to tissue of heart 2 such that the electrode may establish electrical communication with the tissue for mapping.

The medical lead system 100 can be configured to conduct contact-based and/or substantially contact-less pace mapping of heart 2 using the electrode. With the distal balloon 124 in the inflated configuration, a clinician may navigate lead body distal portion 112 to potential implantation sites (e.g., target site 114, 116) and evaluate the adequacy of each potential site through observation of a signal (e.g., ECG and/or EGM signals) generated by the heart 2 as the electrode electrically communicates with the tissue. When the clinician determines that the medical lead system 100 has located an adequate target site, the clinician may deflate the distal balloon 124 to allow insertion of the fixation member 122 at the target site. The distal balloon 124 can substantially re-establish the deflated configuration when the clinician causes the balloon to deflate.

In one or more embodiments, the medical lead system 100 is configured to monitor the pressure within the interior volume 134 (FIGS. 7-9) defined by the distal balloon 124. For example, the medical lead system 100 can include the pressure sensor 200 that is configured to sense the pressure within the interior volume 134 and communicate a signal indicative of the pressure to processing circuitry of an output device. The medical lead system 100 can be configured such that the clinician can monitor the pressure as the lead body 110 is transited through the vasculature of patient 4 and/or chambers of the heart 2 to provide an indication of the location of the lead body 110 within the patient 4. For example, the signal indicative of the pressure may provide an indication that the lead body 110 has entered a chamber of heart 2.

FIG. 5 is a schematic cross-section view of a portion of the medical lead system 100 in a configuration that might be utilized to deliver the lead body 110 to vasculature or other areas within the patient 4 enroute to positioning the distal portion 112 of the lead body 110 in the vicinity of a target site. FIG. 5 illustrates the lead body 110 positioned within a sheath lumen 126 of a sheath 128. Sheath 128 may be, for example, an introducer sheath, such an introducer sheath configured to provide access to a jugular, innominate, and/or subclavian vein. In one or more embodiments, the sheath 128 is a delivery catheter. The sheath 128 includes an inner wall 127 defining sheath lumen 126 and further includes a sheath opening 130 to sheath lumen 126. The distal balloon 124 is affixed to the distal portion 112 of the lead body 110. The fixation member 122 extends distal (e.g., in the distal direction D) to the distal end 120 of the distal portion 112 of the lead body 110. The inner wall 127, distal portion 112, distal end 120, balloon 124, and fixation member 122 are depicted in dashed lines within the sheath lumen 126.

FIG. 5 illustrates the medical lead system 100 with the distal balloon 124 in the deflated configuration. The medical lead system 100 is configured to translate through the sheath lumen 126 to pass through the sheath opening 130 when the distal balloon 124 is in the deflated configuration. The fixation member 122 mechanically supports the electrode 123 configured to electrically communicate with tissue when positioned in the vicinity of a target site within patient 4, such as target site 114, 116.

FIG. 6 is a schematic cross-section view of the medical lead system 100 in a configuration that might be utilized to position the distal balloon 124 outside of the sheath lumen 126 such that balloon may be expanded enroute to positioning the distal portion 112 of the lead body 110 in the vicinity of a target site within patient 4. FIG. 6 illustrates the distal balloon 124 in the deflated configuration and with a maximum initial dimension D1 (e.g., a diameter) perpendicular to a longitudinal axis L defined by the lead body 110. The medical lead system 100 defines the maximum initial dimension D1 to allow the lead body 110 to translate through the sheath lumen 126 and the sheath opening 130. In one or more embodiments, the lead body 110 includes a marker 132 (e.g., proximal to distal balloon 124) configured to indicate that the distal balloon is distal to sheath opening 130 such that balloon 124 is free to expand without constraint by sheath lumen 126 (e.g., inner wall 127). The marker 132 may be configured to be visible on an imaging system, such as a fluoroscope, ultrasound, or other systems configured to provide images of the medical lead system 100 within the patient 4.

FIG. 7 is a schematic cross-section view of the medical lead system 100 with the distal balloon 124 in the inflated configuration. The distal balloon 124 defines the interior volume 134 configured to contain the inflating medium to cause the balloon to transition from the deflated configuration of FIGS. 5-6 to the inflated configuration depicted in FIG. 7. In one or more embodiments, the interior volume 134 is bound at least in part by an inner surface 136 of the distal balloon 124 and the exterior surface 138 of the lead body distal portion 112. The lead body 110 defines the lumen 142 that is configured to provide the inflating medium to the interior volume 134.

In the inflated configuration, the distal balloon 124 defines a maximum expanded dimension D2 (e.g., a diameter) perpendicular to longitudinal axis L. The maximum expanded dimension D2 of the inflated configuration is greater than the maximum initial dimension D1 of the deflated configuration. In the inflated configuration, the distal balloon 124 extends distal to the distal end 120 of the lead body 110, with a portion of the fixation member 122 extending distal to the balloon. The distal balloon 124 can substantially surround the longitudinal axis L in the inflated configuration, such that balloon 124 substantially forms a bumper circumferentially around fixation member 122. In one or more embodiments, the distal balloon 124 defines a substantially toroidal shape surrounding the distal portion 112 and distal end 120 of the lead body 110 when the balloon is in the inflated configuration. The distal balloon 124 can be configured such that the distal portion 112 extends at least partially within a hole defined by the substantially toroidal shape. In one or more embodiments, the fixation member 122 is configured to extend at least partially through the hole defined by the substantially toroidal shape.

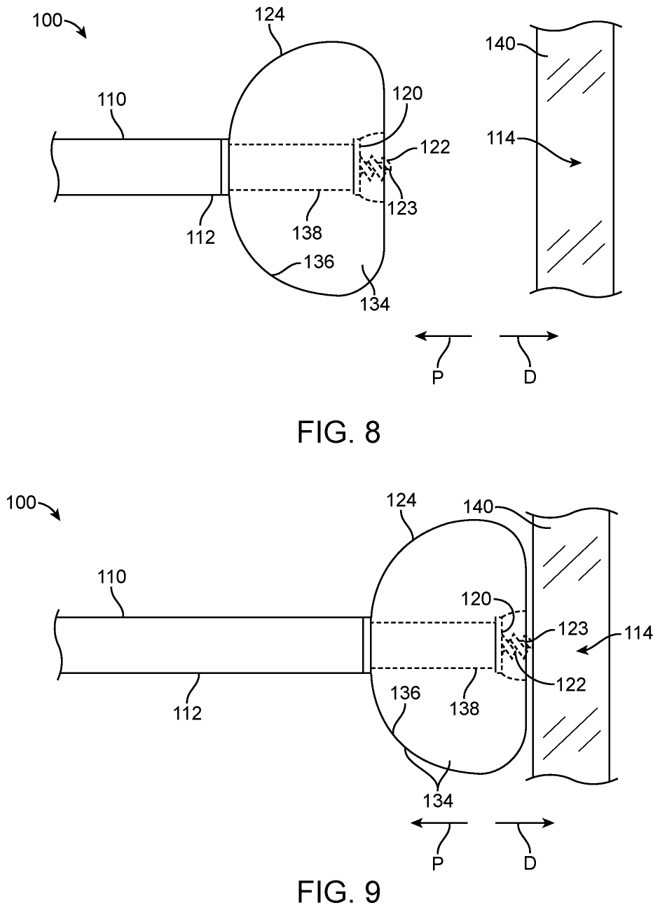

FIG. 8 illustrates the medical lead system 100 with the distal balloon 124 in the inflated configuration approaching a tissue wall 140. The tissue wall 140 can be, for example, a septal wall within the heart 2 (FIG. 1). The distal balloon 124 substantially surrounds the longitudinal axis L and extends distal to the distal end 120 of the lead 106 such that the distal balloon 124 forms a bumper around the fixation member 122 to minimize and/or eliminate physical interference between the fixation member 122 and other anatomical structures within the patient 4 enroute to the tissue wall 140. FIG. 9 illustrates the medical lead system 100 with the distal balloon 124 in the inflated configuration and having positioned the electrode 123 in sufficient proximity to electrically communicate with the tissue wall 140. The medical lead system 100 can be configured such that the electrode 123 electrically communicates with the tissue wall 140 by substantially contacting the tissue wall and/or electrically communicating with the tissue wall in a substantially contactless manner (e.g., using the conductivity of an intervening blood volume). The electrode 123 can be electrically connected to one or more conductors 148 that extend through the lead body 110 and are electrically connected to the processing circuitry 24 of the medical device 12 (FIG. 2). With the electrode 123 in sufficient proximity to the tissue wall 140, a clinician may navigate the distal portion 112 of the lead body 110 to potential implantation sites and evaluate the adequacy of each potential site through observation of a signal relayed from electrode 123 to the circuitry 24. Hence, as illustrated in FIG. 9, the clinician can perform cardiac mapping using the electrode 123 while the distal balloon 124 is in the inflated configuration. The cardiac mapping can occur as the distal balloon 124 substantially shrouds the fixation member 122 to reduce and/or eliminate physical interferences between the fixation member and surrounding anatomical structures within the patient 4. When the clinician determines that the medical lead system 100 has located an adequate target site, the clinician may deflate the distal balloon 124 to allow insertion of the fixation member 122 at the target site.

FIG. 10 illustrates a schematic cross-section view of a portion of the medical lead system 100 with the distal balloon 124 in the deflated configuration. FIG. 11 is a schematic cross-section view of the portion of the medical lead system 100 with the distal balloon 124 in the inflated configuration. The cross-sections of FIG. 10 and FIG. 11 are taken with a cutting plane parallel to the page and longitudinal axis L.

The distal portion 112 of the lead body 110 can define the lumen 142 extending at least partially through the distal portion 112 of the lead body 110. In one or more embodiments, the longitudinal axis L extends through the lumen 142 and intersects the distal end 120 of the lead 106. Lead body 110 (e.g., lead body distal portion 112) can include a wall 144 that defines an inner surface 146, with the inner surface defining the lumen 142. Wall 144 may further define the exterior surface 138 opposite the inner surface 146. In one or more embodiments, the lumen 142 extends to a proximal opening 139 (FIG. 1) defined in a proximal portion of the lead body 110. The lead body 110 can be configured such that the proximal opening 139 is extracorporeal to the patient when the lead body 110 extends through vasculature of patient 4 (FIG. 1). In one or more embodiments, the lead body 110 is configured such that proximal opening 139 is extracorporeal to the patient when the distal portion 112 is positioned within a chamber of the heart 2 (FIG. 1).

The medical lead system 100 can include a conductor 148 in electrical communication with the electrode 123. In one or more embodiments, the fixation member 122 defines the electrode 123, and the conductor 148 electrically communicates with the electrode 123 via the fixation member 122. For example, the fixation member 122 can include a conductive material covered by an insulative layer, and the electrode 123 can be a portion of the conductive material where the insulative layer is removed.

The conductor 148 can extend through the inner lumen 142, although this is not required. In one or more embodiments, the conductor 148 is configured to electrically communicate with the circuitry 24 of the implantable medical device 12 to deliver therapy to the patient 4 using the electrode 123. In one or more embodiments, the conductor 148 is configured to extend to a position extracorporeal to the patient when the lead body 110 extends through vasculature of the patient 4 (FIG. 1), and/or when the lead body distal portion 112 is positioned within the chamber of the heart 2 (FIG. 1).

In one or more embodiments, the medical lead system 100 is configured such that the lumen 142 is fluidly isolated from the patient 4 when the lead body 110 extends through vasculature of the patient 4 (FIG. 1). The medical lead system 100 can be configured such that lumen 142 is fluidly isolated from the distal end 120 of the lead body 110 when the distal end is within vasculature of patient 4. In one or more embodiments, the medical lead system 100 includes a stop 150 configured to fluidly isolate the lumen 142 and the distal end 120 of the lead body 110 when the distal end 120 is within vasculature of the patient 4. In one or more embodiments, the stop 150 is configured to mechanically support the fixation member 122. Hence, the inner lumen 142 can be configured to such that the lumen 142 is fluidly coupled to a proximal opening (e.g., inlet 102 of FIG. 2) and fluidly isolated from the distal end 120 when the distal end is within vasculature of the patient 4.

The distal balloon 124 can include a balloon body 152 configured to elastically expand when the balloon transitions from the deflated configuration to the inflated configuration (e.g., when an inflating medium is provided to the interior volume 134). The balloon body 152 can define the balloon inner surface 136. In one or more embodiments, the balloon body 152 extends at least partially around a perimeter defined by distal exterior surface 138 (e.g., a perimeter perpendicular to longitudinal axis L). In one or more embodiments, the balloon body 152 extends substantially completely around the perimeter defined by the distal exterior surface 138.

The balloon body 152 (e.g., balloon inner surface 136) and a portion of the distal exterior surface 138 can define the interior volume 134. In one or more embodiments, the distal portion 112 of the lead body 110 defines the distal exterior surface 138 to include a first exterior surface 138a and a second exterior surface 138b, with the first exterior surface proximal to second exterior surface 138b. The balloon body 152 and the second exterior surface 138b can define at least a portion of or in some cases substantially all of the inner boundary of the interior volume 134. In one or more embodiments, the second exterior surface 138b extends substantially from the first exterior surface 138b to the distal end 120.

The medical lead system 100 includes one or more structures defining the lumen 142 configured to deliver the inflating medium to the interior volume 134 to cause inflation of the distal balloon 124. In one or more embodiments, the lead body 110 defines the lumen 142 configured to deliver the inflating medium. Lead distal portion 112 can include one or more side lumens such as side lumen 154 and side lumen 156 configured to fluidly couple the lumen 142 and the interior volume 134 such that the inflating medium provided to the lumen can flow to the interior volume 134 via side lumens 154, 156. In one or more embodiments, at least one of the side lumens 154, 156 define the outlet 118 of the lumen 142.

As described herein, the distal balloon 124 is configured to expand radially outward from the distal exterior surface 138 (e.g., second exterior surface 138b) and extend distally beyond the distal end 120 when the balloon inflates from the deflated configuration (FIG. 10) to the inflated configuration (FIG. 11). In one or more embodiments, the balloon body 152 is affixed to the exterior surface 138 of the lead body 110 at least at a location 158 and is configured to extend distal to the location when the balloon is in the inflated configuration. In one or more embodiments, the balloon body 152 is affixed to the exterior surface 138 at a first location 160 and a second location such as location 158, with the first location 160 proximal to the second location 158. The balloon body 152 can define a midpoint M between (e.g., substantially halfway between) the first location 160 and the second location 158 and separating a proximal balloon portion 162 and a distal balloon portion 164. The proximal balloon portion 162 can extend between the first location 160 and the midpoint M, and the distal balloon portion 164 can extend between midpoint M and the second location 158. The distal balloon 124 can be configured such that distal balloon portion 164 is distal to the second location 158 when the balloon is in the inflated configuration. In one or more embodiments, the distal balloon 124 (e.g., balloon body 152) is configured to be proximal to the distal end 120 of the lead body 110 when the balloon is in the deflated configuration. The distal balloon 124 (e.g., balloon body 152) may be configured to expand in a direction perpendicular to the longitudinal axis L when the balloon 124 expands radially outward from distal exterior surface 138.

Fluidly connected to the inlet 102 of the lumen 142 of the lead body 110 is the pressure sensor 200. Such sensor 200 can include any suitable type of sensor that is configured to sense a pressure within the interior volume 134 of the distal balloon 124 and communicate a signal indicative of the pressure to the clinician. In one or more embodiments, the pressure sensor 200 includes at least one of a pressure transducer, an audible alarm, an indicator light, or a mechanical switch. For example, in one or more embodiments, the pressure sensor 200 can include an indicator light that is configured to emit light having a first wavelength when the distal balloon 124 is in the deflated configuration and emit light having a second wavelength when the distal balloon is in the inflated configuration. In one or more embodiments, the first wavelength is different from the second wavelength. In embodiments where the pressure sensor 200 includes a mechanical switch, the signal provided to the clinician can include a change of shape of the mechanical switch. For example, in one or more embodiments, the pressure sensor 200 can include a diaphragm that can provide visual confirmation of internal pressurization of the distal balloon 124 or an audible snape or click that can be associated with the diaphragm moving through a toggle position as the distal balloon is pressurized.

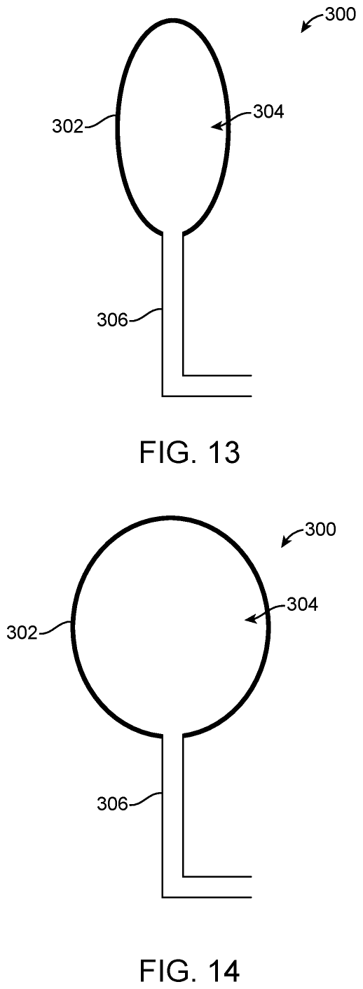

FIGS. 13-14 are schematic cross-section views of one embodiment of a pressure sensor 300 that can be utilized with the system 10 of FIGS. 1-11. All design considerations and possibilities described herein regarding the pressure sensor 200 of FIG. 2 apply equally to the pressure sensor 300 of FIGS. 13-14. The pressure sensor 300 includes an indicator balloon 302 that can be connected to the inlet 102 of the lumen 142 of the lead body 110 by a conduit 306. The indicator balloon 302 can define a deflated configuration (FIG. 13) and an inflated configuration (FIG. 14). The indicator balloon 302 is in the deflated configuration when the distal balloon 124 is in the deflated configuration. Further, the indicator balloon 302 is in the inflated configuration when the distal balloon 124 is in the inflated configuration.

In one or more embodiments, an interior volume 304 of the indicator balloon 302 can be representative of the interior volume 134 of the distal balloon 124. For example, a suitable transfer function can be utilized that correlates the interior volume 304 of the indicator balloon 302 to the interior volume 134 of the distal balloon 124. Such interior volume 304 of the indicator balloon 302 can be correlative of a pressure in the fluid path 206 formed by the sensor 300, the lumen 142 of the lead 106, the inflation mechanism 202 when it is fluidly connected to the inlet 102 of the lead, and the distal balloon 124. As a result, the pressure within the indicator balloon 302 can corelate to the pressure within the distal balloon 124. The indicator balloon 302 can provide a signal that can be communicated to the clinician that is indicative of the pressure within the interior volume of the distal balloon 124. In one or more embodiments, such signal can be provided by the value of the interior volume of the indicator balloon 302, i.e., the size of the indicator balloon as viewed by the clinician.

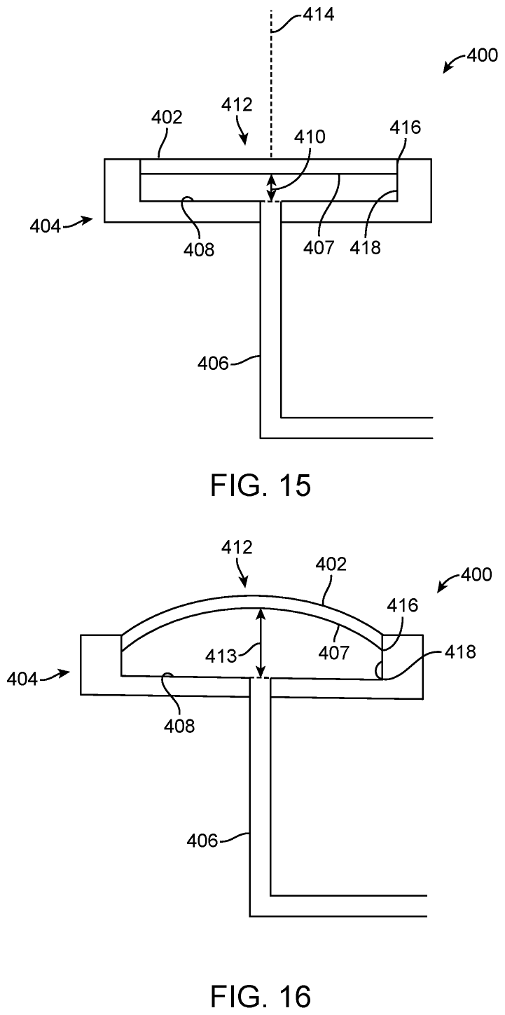

FIGS. 15-16 are schematic cross-section views of another embodiment of a pressure sensor 400. All design considerations and possibilities described herein regarding the pressure sensor 200 of FIGS. 2 and the pressure sensor 300 of FIGS. 13-14 apply equally to the pressure sensor 400 of FIGS. 15-16. The pressure sensor 400 includes a disk 402 and a base 404 configured to receive the disk. The base 404 can be connected to the inlet 102 of the lumen 142 of the lead body 110 by a conduit 406. A major surface 407 of the disk 402 faces a major surface 408 of the base 404. The pressure sensor 400 is configured to provide a signal that includes a first configuration (FIG. 15) and a second configuration (FIG. 16) of the disk 402. A first distance 410 between a center portion 412 of the major surface 407 of the disk 402 and the major surface 408 of the base 404 when the disk is in the first configuration as measured along a normal 414 to the major surface of the disk and the major surface the base is less than a second distance 413 between the center portion of the major surface of the disk and the major surface of the base when the disk is in the second configuration. The disk 402 is in the first configuration when the distal balloon 124 is in the deflated configuration and in the second configuration when the distal balloon is in the inflated configuration.

The disc 402 can take any suitable shape or shapes in a plane parallel to the major surface 407 of the disk, e.g., elliptical, rectangular, or polygonal shape. Further, the base 404 can take any suitable shape or shapes in the plane parallel to the major surface 407 of the disk 402. The disk 402 can be connected to the base 404 using any suitable technique. For example, the disk 402 can be constrained along an edge 416 within a notch or slot (not shown) in a side wall 418 of the base 404.

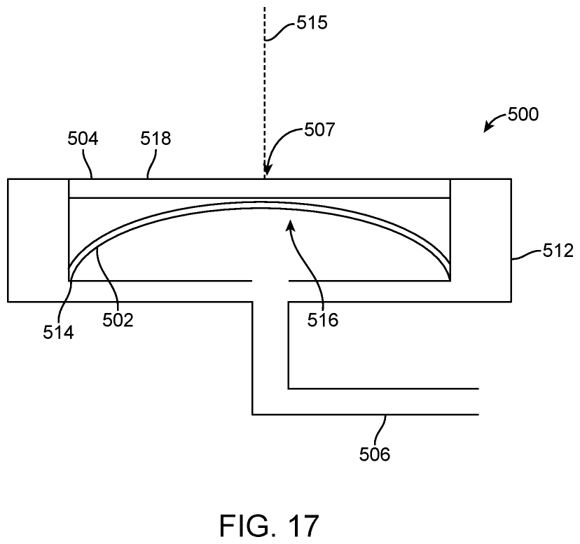

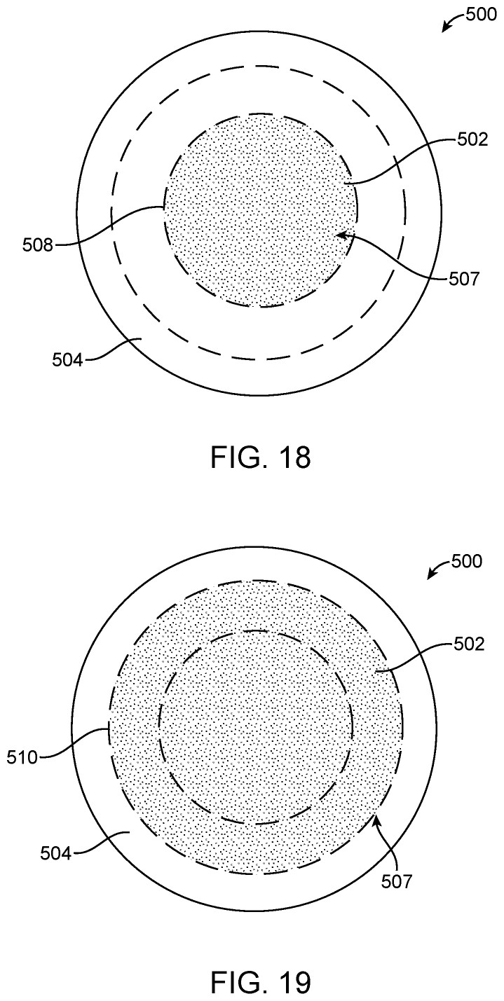

FIGS. 17-19 are various views of another embodiment of a pressure sensor 500. All design considerations and possibilities described herein regarding the pressure sensor 200 of FIG. 2, the pressure sensor 300 of FIGS. 13-14, and the pressure sensor 400 FIGS. 15-16 apply equally to the pressure sensor 500 of FIGS. 17-19. The pressure sensor 500 includes a colored diaphragm 502 and a frosted lens 504. The colored diaphragm 502 and the frosted lens 504 can be disposed in a base 512 that is fluidly connected to the inlet 102 of the lumen 142 by a conduit 506. The color diaphragm 502 is configured to move in a direction along a normal 515 to a major surface 518 of the frosted lens 504. A contact area 507 (FIGS. 18-19) between the colored diaphragm 502 and the frosted lens 504 corresponds to a pressure of the interior volume 134 of the distal balloon 124. For example, as shown in FIG. 18, the contact area 507 is within a first indicator zone 508. Such indicator zone 508 can correlate to a deflated configuration of the distal balloon 124. As shown in FIG. 19, the contact area 507 fills a second indicator zone 510 that has a greater area than the first indicator zone 508 FIG. 18. The second indicator zone 510 can correlate to an inflated configuration of the distal balloon 124.

The colored diaphragm 502 and the frosted lens 504 can be disposed in the base 512 using any suitable technique. As shown in FIG. 17, the colored diaphragm 502 is fixed along an edge 514 such that the edge is constrained while a center portion 516 is free to move in relation to the pressure in the interior volume 134 of the distal balloon 124 and contact the frosted lens 504 to form the contact area 507, which is indicative of the pressure in the interior volume 134 of the distal balloon 124. The colored diaphragm 502 and the frosted lens 504 can take any suitable shape or shapes in a plane orthogonal to the normal 515 of a major surface 518 of the lens as shown in FIG. 17. As the center portion 516 is displaced by the pressure within the air flow path, the center portion is forced into intimate contact with the major surface 518 of the frosted lens 504, thereby substantially changing the visual appearance of the lens. As the pressure within the air flow path decreases, the diaphragm 502 moves in a direction away from the frosted lens 504 such that the visual appearance of the frosted lens returns to its original state.

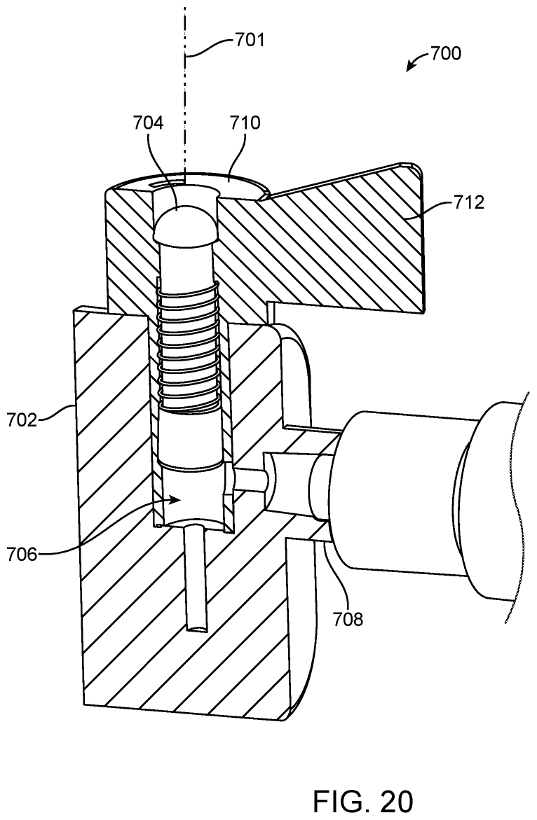

FIG. 20 is a schematic cross-section view of another embodiment of a pressure sensor 700. All design considerations and possibilities described herein regarding the pressure sensors 200, 300, 400, and 500 apply equally to the pressure sensor 700 of FIG. 20. In one or more embodiments, the sensor 700 can be considered to be a mechanical switch-type sensor, where a signal communicated to the clinician can include a change of shape of a mechanical switch of the sensor. The sensor 700 includes a body 702 and a spring-loaded plunger 704 disposed within a channel 706 that extends into the body. The channel 706 is fluidly connected to a conduit 708 that can be connected to the inlet 102 of the lumen 142. The spring-loaded plunger 704 is configured to move in a direction along a channel axis 701 along which the channel 706 extends. Pressure within the channel 706 corresponds to pressure within the interior volume 134 of the distal balloon 124 such that when the interior volume reaches a desired pressure, the plunger 704 extends beyond an upper surface 710 of a valve 712 that is connected to the body 702, i.e., the pressure sensor 700 communicates a signal indicative of the pressure to the clinician. In one or more embodiments, the valve 712 can be utilized to control activation of the plunger 704. Further, such valve can be utilized as a deflation switch that can be utilized to at least partially deflate the distal balloon 124.

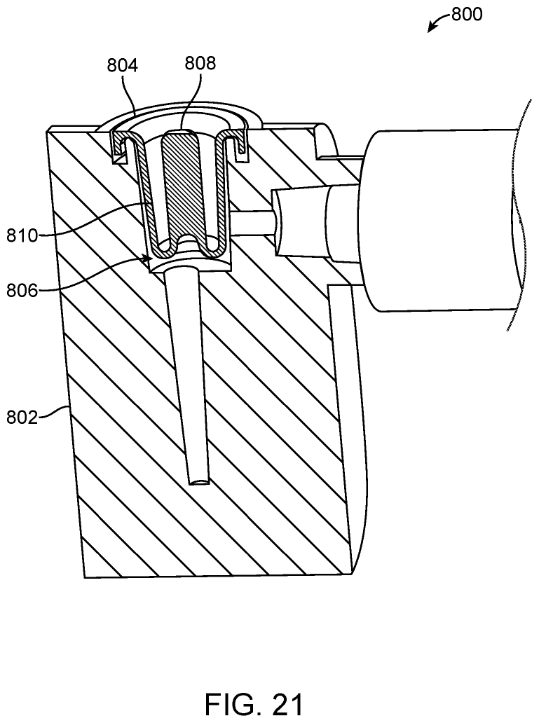

FIG. 21 is a schematic cross-section view of another embodiment of a pressure sensor 800. All design considerations and possibilities described herein regarding the pressure sensors 200, 300, 400, 500, and 700 apply equally to the pressure sensor 800 of FIG. 21. In one or more embodiments, the sensor 800 can be considered to be a mechanical switch-type sensor, where a signal communicated to the clinician can include a change of shape of a mechanical switch of the sensor. The pressure sensor 800 includes a body 802 and a pop-up indicator 804 disposed within a channel 806 of the body. The indicator 804 includes sidewalls 810 and a stem 808 connected to the sidewalls. When fluid pressure is applied to the valve 800, the sidewalls 810 prolapse or invert, thereby causing the stem 808 to protrude beyond the body 802. Once pressure is removed from the valve 800, the sidewalls 810 return to a relaxed state, which causes the stem 808 to retract back into the body 802. The indicator 802 can be correlated to the pressure of the interior volume 134 of the distal balloon 124 such that the stem 808 extends beyond the body 802 when the interior volume is at a desired pressure. As a result, the pressure sensor 800 senses the pressure within the interior volume 134 of the distal balloon 124 and communicates a signal indicative of the pressure to the clinician.

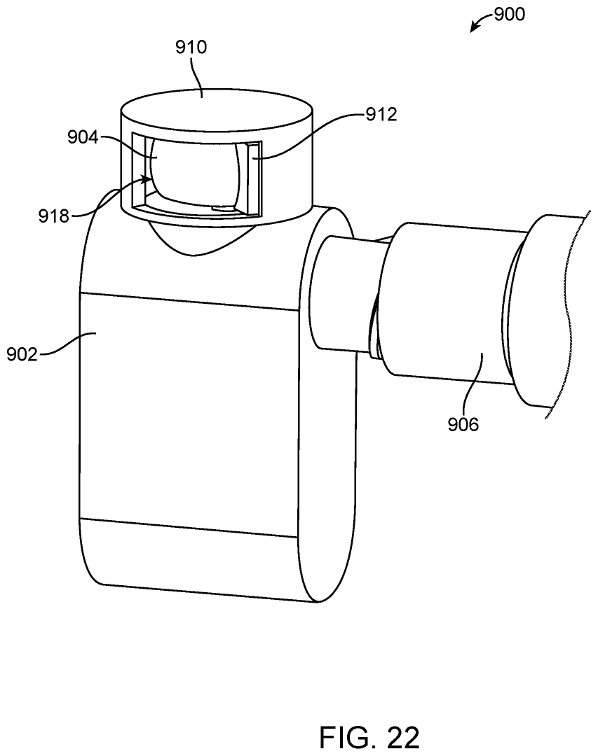

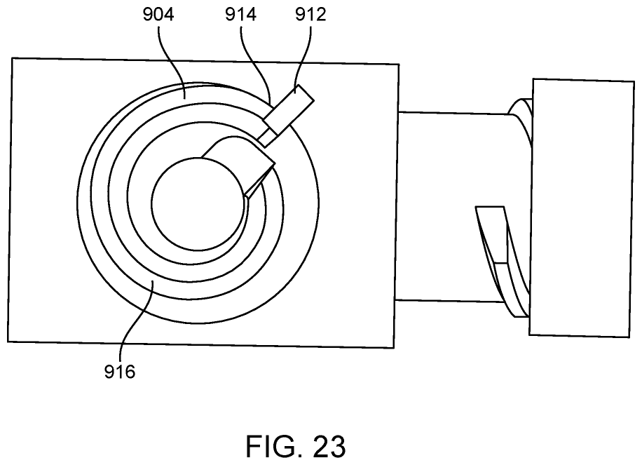

FIG. 22 is a schematic cross-section view of another embodiment of a pressure sensor 900. All design considerations and possibilities described herein regarding the pressure sensor 200, 300, 400, 500, 700, and 800 apply equally to the pressure sensor 900 of FIG. 22. In one or more embodiments, the sensor 900 can be considered to be a mechanical switch-type sensor, where a signal communicated to the clinician can include a change of shape of a mechanical switch of the sensor. The sensor 900 includes a body 902 and a tube 904 disposed within a housing 910 that is connected to the body. The tube 904 is fluidly connected to the inlet 102 of the lumen 142 by a conduit 906. As can be seen in FIG. 23, which is a schematic top plan view of the pressure sensor 900 of FIG. 22 with the housing 910 removed for clarity, an indicator tab 912 can be connected to an end 914 of the tube 904. As shown, the tube 904 can be in a coiled configuration and flattened or deflated. When fluid pressure is applied to the sensor 900, walls 916 of the tube 904 can expand as the tube inflates. Such expansion of the walls 916 causes the tube 904 to uncoil, thereby moving the indicator tab 912 within a window 918 of the housing 910. A position of the indicator tab 912 within the window 918 can be correlated with a pressure of the interior volume 134 of the distal balloon 124. As a result, movement of the indicator tab 912 indicates to the clinician a change in pressure of the interior volume 134 of the distal balloon 124, i.e., the pressure sensor 900 communicates a signal indicative of the pressure to the clinician.

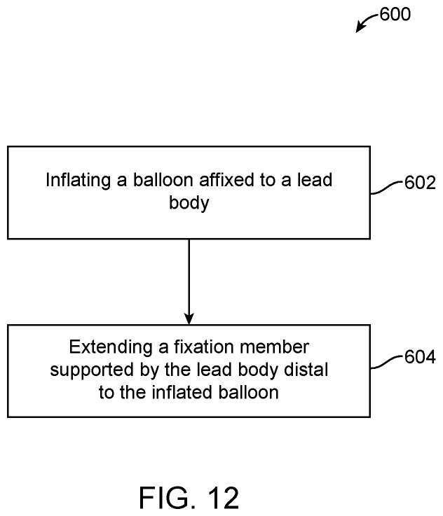

A method 600 for positioning a lead body 110 within a patient 4 is illustrated in FIG. 12. Although the method 600 is described in regard to medical lead system 100, the method can be utilized with any suitable medical lead system.

The method includes inflating the distal balloon 124 from the deflated configuration to the inflated configuration at 602. The method can include expanding the distal balloon 124 radially outward from the exterior surface 138 of the distal portion 112 of the lead body 110 when the balloon inflates from the deflated configuration to the inflated configuration. In one or more embodiments, the method includes positioning the lead body 110 within vasculature of the patient 4 prior to inflating the distal balloon 124 from the deflated configuration to the inflated configuration.

In one or more embodiments, the method further includes issuing an inflating medium into the interior volume 134 defined by the distal balloon 124 to inflate the balloon from the deflated configuration to the inflated configuration. The method can include issuing the inflating medium through the lumen 142 that is fluidly coupled to the interior volume 134. In one or more embodiments, the inflating medium issues through the lumen 142 to the interior volume 134 via side lumens 154, 156, i.e., outlet 118.

Balloon body 152 may extend distal to a distal end 120 of the lead body 110 when the distal balloon 124 inflates. In one or more embodiments, the balloon body 152 is affixed to the exterior surface 138 at a location on the exterior surface, and a portion of the balloon body displaces from a position proximal to the location to a position distal to the location when the distal balloon 124 inflates. In one or more embodiments, the balloon body 152 is affixed to the exterior surface 138 at a first location and a second location distal to the first location, and a distal balloon portion 164 is distal to the first location and proximal to the second location when the distal balloon 124 is in the deflated configuration. Distal balloon portion 164 can extend distal to the distal end 120 when the distal balloon 124 inflates.

The method 600 further includes extending the fixation member 122 mechanically supported by the distal portion 112 distally beyond the distal balloon 124 when the balloon is in the inflated configuration at 604. The fixation member 122 can define a helix including a helix distal end, where the helix distal end can extend distally beyond the beyond the distal balloon portion 164 when the distal balloon 124 is in the inflated configuration. The distal balloon 124 can extend radially around a portion of the fixation member 122 when the balloon is in the inflated configuration and fixation member 122 extends distal to the balloon. In one or more embodiments, the distal balloon 124 substantially surrounds a portion of the fixation member 122 when the balloon is in the inflated configuration and the fixation member 122 extends distal to the balloon.

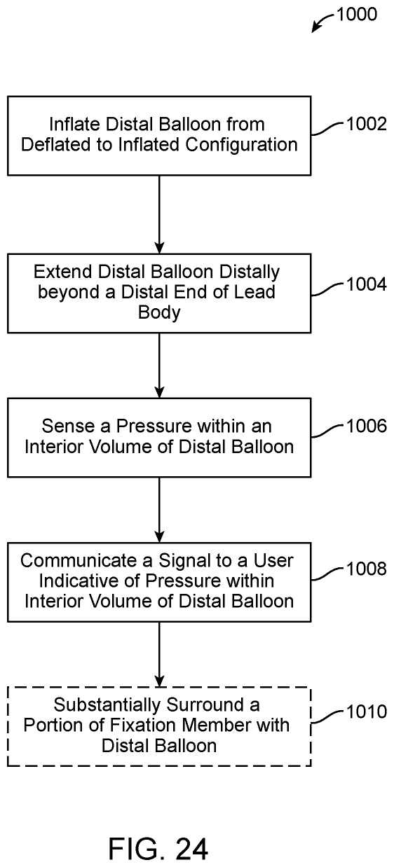

FIG. 24 is a flowchart of one embodiment of a method 1000 of inflating the distal balloon 124 and sensing a pressure within its interior volume 134. Although described in regard to the implantable medical device assembly 10 of FIGS. 1-12, the method 1000 can be utilized with any suitable assembly.

At 1002, the distal balloon 124 is inflated using any suitable technique from a deflated configuration to an inflated configuration by expanding the distal balloon radially outward from the exterior surface 138 of the lead body 110 that is configured to extend through vasculature to a chamber of the heart 4 of the patient 2. The distal balloon 124 can be inflated using any suitable technique, e.g., by utilizing the inflation mechanism 202 of FIG. 2. The distal balloon 124 can be extended distally beyond the distal end 120 of the lead body 110 and proximal to the fixation member 122 when the distal balloon inflates from the deflated configuration to the inflated configuration at 1004. Further, at 1006, the pressure within the interior volume 134 of the distal balloon 124 can be sensed using any suitable technique. For example, in one or more embodiments, the pressure sensor 200 can be utilized to sense the pressure within the interior volume 134 of the distal balloon 124. A signal can be communicated to the clinician at 1008 that is indicative of the pressure within the interior volume 134 of the distal balloon 124 using any suitable technique or techniques. For example, such signal can include the interior volume of the indicator balloon 302 of sensor 300 FIGS. 13-14 that indicates the pressure within the interior volume 134 of the distal balloon 124. In one or more embodiments, a portion of the fixation member 122 can be substantially surrounded with the distal balloon 124 when the distal balloon inflates to the inflated configuration at 1010.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include computer-readable storage media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

1. A medical lead system comprising:
a lead comprising:
a lead body comprising a distal end configured to extend through vasculature to a chamber of a heart of a patient, wherein the lead body further comprises a lumen that extends along the lead body between an inlet adjacent to a proximal end of the lead body and an outlet adjacent to the distal end of the lead body;
a fixation member configured to extend distal to the distal end of the lead body, wherein the fixation member is configured to be secured to tissue of the heart; and
a distal balloon connected to an exterior surface of the distal end of the lead body and fluidly connected to the outlet of the lumen, wherein the distal balloon defines a deflated configuration and an inflated configuration, and further wherein the distal balloon is configured to expand radially outward from the exterior surface of the lead body and extend distally beyond the distal end of the lead body when the distal balloon inflates from the deflated configuration to the inflated configuration; and
a pressure sensor fluidly connected to the inlet of the lumen of the lead body and configured to sense a pressure within an interior volume of the distal balloon and communicate a signal indicative of the pressure to a clinician;
wherein the fixation member is further configured to extend distal to the distal balloon when the distal balloon is in the inflated configuration.

2. The system of claim 1, further comprising an inflation mechanism configured to be fluidly connected to the inlet of the lumen of the lead body and manipulate the distal balloon between the deflated configuration and the inflated configuration.

3. The system of claim 2, wherein the inflation mechanism comprises a syringe or a pump.

4. The system of claim 1, wherein the pressure sensor comprises an indicator balloon that defines a deflated configuration and an inflated configuration, wherein the indicator balloon is in the deflated configuration when the distal balloon is in the deflated configuration, and further wherein the indicator balloon is in the inflated configuration when the distal balloon is in the inflated configuration.

5. The system of claim 1, wherein the pressure sensor comprises a disk and a base configured to receive the disk, wherein a major surface of the disk faces a major surface of the base, wherein the signal comprises a first configuration and a second configuration of the disk, wherein a first distance between a center portion of the major surface of the disk and the major surface of the base when the disk is in the first configuration as measured along a normal to the major surface of the disk and the major surface of the base is less than a second distance between the center portion of the major surface of the disk and the major surface of the base when the disk is in the second configuration, wherein the disk is in the first configuration when the distal balloon is in the deflated configuration and in the second configuration when the distal balloon is in the inflated configuration.

6. The system of claim 1, wherein the pressure sensor comprises a pressure transducer.

7. The system of claim 1, wherein the pressure sensor comprises an indicator light, wherein the indicator light is configured to emit light comprising a first wavelength when the distal balloon is in the deflated configuration and emit light comprising a second wavelength when the distal balloon is in the inflated configuration.

8. The system of claim 1, wherein the pressure sensor comprises a colored diaphragm and a frosted lens, wherein the colored diaphragm is configured to move in a direction normal to the diaphragm and frosted lens, wherein a contact area between the colored diaphragm and the frosted lens corresponds to a pressure within the interior volume of the distal balloon.

9. An implantable medical device assembly comprising:
an implantable medical device comprising a housing and an electronics module disposed within the housing and comprising a power source and circuitry electrically connected to the power source; and
a lead system comprising:
a lead comprising:
a lead body comprising a distal end configured to extend through vasculature to a chamber of a heart of a patient, wherein the lead body comprises a lumen that extends along the lead body between an inlet adjacent to a proximal end of the lead body and an outlet adjacent to the distal end of the lead body, wherein at least a proximal portion of the lead body is configured to be inserted into a lead bore of the housing;
a fixation member configured to extend distal to the distal end of the lead body, wherein the fixation member is configured to secure to tissue of the heart; and
a distal balloon connected to an exterior surface of the distal end of the lead body and fluidly connected to the outlet of the lumen, wherein the distal balloon defines a deflated configuration and an inflated configuration, and further wherein the distal balloon is configured to expand radially outward from the exterior surface of the lead body and extend distally beyond the distal end of the lead body when the distal balloon inflates from the deflated configuration to the inflated configuration; and
a pressure sensor fluidly connected to the inlet of the lumen of the lead body and configured to sense a pressure within an interior volume of the distal balloon and communicate a signal indicative of the pressure to a clinician;
wherein the fixation member is configured to extend distal to the distal balloon when the distal balloon is in the inflated configuration.

10. The assembly of claim 9, wherein the lead bore of the implantable medical device comprises a contact that is electrically connected to the circuitry of the electronics module by a conductor, wherein a lead contact of the lead is configured to be electrically connected to the contact of the lead bore when the at least a portion of the lead is disposed within the lead bore.

11. The assembly of claim 9, wherein the implantable medical device is one of an implantable defibrillator or a neuromodulation device.

12. The assembly of claim 9, further comprising an inflation mechanism configured to be fluidly connected to the inlet of the lumen and manipulate the distal balloon between the deflated configuration and the inflated configuration.

13. The assembly of claim 12, wherein the pressure sensor is connected to the inflation mechanism.

14. The assembly of claim 9, wherein the pressure sensor comprises an indicator balloon that defines a deflated configuration and an inflated configuration, wherein the indicator balloon is in the deflated configuration when the distal balloon is in the deflated configuration, and further wherein the indicator balloon is in the inflated configuration when the distal balloon is in the inflated configuration.

15. The assembly of claim 9, wherein the pressure sensor comprises a disk and a base configured to receive the disk, wherein a major surface of the disk faces a major surface of the base, wherein the disk comprises a first configuration and a second configuration, wherein a first distance between a center portion of the major surface of the disk and the major surface of the base when the disk is in the first configuration as measured along a normal to the major surface of the disk and the major surface of the base is less than a second distance between the center portion of the major surface of the disk and the major surface of the base when the disk is in the second configuration, wherein the disk is in the first configuration when the distal balloon is in the deflated configuration and in the second configuration when the distal balloon is in the inflated configuration.

16. The assembly of claim 9, wherein the pressure sensor comprises a pressure transducer.

17. The assembly of claim 9, wherein the pressure sensor comprises an audible alarm.

18. The assembly of claim 9, wherein the pressure sensor comprises an indicator light, wherein the indicator light is configured to emit light comprising a first wavelength when the distal balloon is in the deflated configuration and emit light comprising a second wavelength when the distal balloon is in the inflated configuration.

19. The assembly of claim 9, wherein the pressure sensor comprises a colored diaphragm and a frost lens, wherein the colored diaphragm is configured to move in a direction normal to the diaphragm and frosted lens, wherein an area of contact between the colored diaphragm and the frosted lens corresponds to the pressure of the distal balloon.

20. The assembly of claim 9, wherein the pressure sensor comprises a mechanical switch, wherein the signal comprises a change of shape of the mechanical switch.
